(19) United States
(12) Patent Application Publication
(10) Pub. No.: US 2025/0114611 A1
(43) Pub. Date: Apr. 10, 2025

(54) MRI DETECTION SYSTEM FOR IMPLANTABLE MEDICAL DEVICE

(71) Applicant: Axonics, Inc., Irvine, CA (US)

(72) Inventors: Faizal ABDEEN, Irvine, CA (US); Guangqiang JIANG, Irvine, CA (US); Yuqing WAN, Irvine, CA (US)

(73) Assignee: Axonics, Inc., Irvine, CA (US)

(21) Appl. No.: 18/908,733

(22) Filed: Oct. 7, 2024

Related U.S. Application Data
(60) Provisional application No. 63/542,972, filed on Oct. 6, 2023.

(51) Int. Cl. A61N 1/36; A61N 1/378

(52) U.S. Cl. CPC A61N 1/36146; A61N 1/3787

(57) ABSTRACT

An MRI detection system for an AIMD. The detection system includes utilizing monitoring or detecting a signal from a magnetic field sensitive element. The magnetic field sensitive element may include at least one of an inductor within the IPG, a reed switch, and Hall sensor. The magnetic field sensitive element may assist the AIMD in detecting an MRI to trigger an MRI safe mode in order to prevent adverse effects when an MRI scan is performed.

### CROSS-REFERENCE TO RELATED APPLICATION

This application claims the benefit of U.S. Provisional Patent Application No. 63/542,972, filed Oct. 6, 2023, which is incorporated by reference herein in its entirety.

### GENERAL DESCRIPTION

This application relates to a magnetic resonance imaging (MRI) detection system for an active implantable medical device (AIMD). Specifically, an MRI detection system used to prevent or minimize the risk associated with heating and/or stimulation anomalies when a patient with an AIMD is subject to an MRI scan. The disclosed system may be configured to prevent or reduce the risk of any damage to the AIMD resulting from an MRI scan.

Sacral Neuromodulator (SNM) is an established therapy that provides a safe, effective, reversible, and long-lasting treatment option for the management of urinary urge incontinence, urgency-frequency, and non-obstructive urinary retention. SNM therapy involves the use of mild electrical pulses to stimulate the sacral nerves located in the lower back. Electrodes are placed next to a sacral nerve, usually at the S3 level, by inserting the electrode leads into the corresponding foramen of the sacrum. The electrodes are inserted subcutaneously and are subsequently attached to an AIMD (e.g., an implantable pulse generator (IPG)), also referred to herein as an “implantable neurostimulator” or a “neurostimulator.” SNM has also been approved to treat chronic fecal incontinence in patients who have failed or are not candidates for more conservative treatments.

Magnetic resonance imaging (MRI) is a non-invasive medical imaging technique that uses a strong magnetic field and radio waves to create detailed images of the body's internal structures. MRI systems (typically a machine) create a strong magnetic field that aligns the magnetic moments of the hydrogen atoms in the body's tissues. A radio frequency pulse is then applied to the body, which causes the hydrogen atoms to absorb energy and move out of their aligned state. As the radio pulse subsides, the absorbed energy is released and the hydrogen atoms return to their aligned state which releases energy in the form of radio waves. The MRI machine detects and processes these radio waves to create detailed images of the body. Both the magnetic field and the radio waves may affect the AIMD adversely.

There have been efforts by different manufacturers in order to minimize any adverse effects of an MRI scan on the operation of an AIMD. For example, the AIMD may be configured to be in a dedicated MRI mode to reduce or prevent any effect on the AIMD. The MRI mode may include completely ceasing stimulation while the MRI scan is performed, or adjusting the operation of the AIMD so that the stimulation would not be affected by the MRI, including in certain situations disabling certain functions of the AIMD, such as sensing. Certain existing systems require that a healthcare professional manually configure the AIMD prior to an MRI. There are also systems that include an AIMD that is configured to automatically detect an MRI scan. These AIMD devices generally contain different MRI safe therapy modes which may be pre-selected by the healthcare professional and stored in the AIMD. Depending on the type of MRI scan (e.g. 1.5T and 3T) that is detected, the AIMD will automatically activate the pre-selected and stored therapy mode in order to prevent the MRI scan from having an adverse effect on the operation of the AIMD. These solutions, however, require a separate sensor to perform such detection and also typically require a larger AIMD to accommodate the additional sensor and any needed circuitry. The healthcare community desires smaller not larger AIMDs in order to improve patient comfort and the case of implantation. Thus, there remains a need for improved MRI detection in AIMDs.

### SUMMARY

The embodiments disclosed herein relate to MRI detection systems that improve on previous MRI detection mechanisms. Embodiments disclosed herein include utilizing already existing ferromagnetic cores of inductors, charging coils within the AIMD, and or separate dedicated inductors for sensing magnetic fields. By monitoring the electrical properties (e.g., inductance or voltage) of an inductor via a circuit, the disclosed system may provide trigger signals in response to one or more of the electrical properties crossing a threshold. The presence of a magnetic field of an MRI may saturate the ferromagnetic cores of the inductors, thereby decreasing magnetic permeability (e.g., ability to store magnetic flux) and, thus, decreasing the inductance of the inductors. When an inductor is saturated, its ability to store energy in a magnetic field is limited. As a result, the inductor's inductance will decrease, as the magnetic field can no longer increase in proportion to the current. Therefore, the inductor's ability to filter or regulate current will be reduced, and the inductor may behave like a resistor rather than an inductor which can lead to a reduction in voltage across the inductor.

Other embodiments include utilizing a reed switch, a magneto resistive sensor, or a Hall sensor to sense the magnetic field. The reed switch is an electromechanical switching device consisting of two ferromagnetic blades that are sealed in a glass envelope. When a magnet approaches these blades, the two blades pull toward one another. Once touching, the blades close the normally open contacts, allowing electricity to flow through the two blades. By monitoring the electrical signal downstream of the reed switch, one can detect the presence of a magnetic field from an MRI. Hall sensors are magnetic sensors that detect the presence and magnitude of a magnetic field using the Hall effect. The Hall effect is a phenomenon in which a voltage is generated across a conductor when a magnetic field is applied perpendicular to the direction of current flow. Hall effect sensors are activated by a magnetic field, and they output an electrical signal proportional to the intensity of the magnetic field around them. The output voltage of a Hall sensor is directly proportional to the strength of the field. By monitoring the output signal from the Hall sensor, we can detect the presence of a magnetic field (e.g., an MRI machine).

The magneto resistive sensor relies upon the detection of a change of resistance caused by the influence of the magnetic field generated by an MRI scan. A magneto resistive sensor may be configured to highly sensitive to changes in the magnetic field. Magneto sensors may be considered to be quite suitable for use in MRI detection in an AIMD because the high sensitivity, low power consumption and larger detection range than more traditional sensors, such as Hall effect sensors.

The advantages of the embodiments discussed above is that it allows the AIMD to safely enter the MRI mode if the patient forgets to modify or turn off the stimulator before entering the MRI scanner and will also allow healthcare professionals to perform emergency scans to patients without prior knowledge of the presence of the implanted device. Additionally, MRI process may be simplified for patients with an AIMD. Patients that utilize an AIMD system that includes the disclosed MRI detection system may benefit from a simplified MRI process that allows for direct MRI procedures, without the need for manual interactions with an AIMD using an external controller, such as a patient remote control and or a clinical programmer.

### BRIEF DESCRIPTION OF THE DRAWINGS

FIG. 1 shows an example of an implantable neurostimulation device having an implantable stimulation lead

FIG. 2 shows an exemplary section view of the implantable neurostimulation device shown in FIG. 1

FIG. 3 shows an exemplary stimulation architecture of the implantable neurostimulation device.

FIG. 4 shows an exemplary MRI detection circuit.

FIG. 5 shows an exemplary flowchart of an MRI detection system.

FIG. 6 shows an exemplary MRI detection circuit according to another embodiment.

FIG. 7 shows an exemplary MRI detection circuit according to another embodiment.

FIG. 8 shows an exemplary MRI detection system according to another embodiment.

FIG. 9 shows an exemplary MRI detection system according to another embodiment.

### DETAILED DESCRIPTION

The particulars shown herein are by way of example and for purposes of illustrative discussion of the disclosed embodiments and are presented to provide a readily understood description of the principles and conceptual aspects. In this regard, no attempt is made to show structural details in more detail than is necessary for a fundamental understanding, and the description taken together with the drawings make apparent to those skilled in the art how the disclosed devices and methods may be embodied in practice.

In one embodiment, an implantable medical device configured to provide electrical stimulation to a patient is disclosed. The device comprising a rechargeable energy storage device, a charging coil configured to receive energy to recharge the rechargeable energy storage device, a controller configured to control a stimulation parameter of the device, an MRI detecting circuit electrically connected to the charging coil, wherein the controller is configured to receive a signal from the MRI detecting circuit. The controller is configured to change the stimulation parameter of the device when the controller detects the signal from the MRI detecting circuit, wherein the signal representative of an inductance change of the charging coil due to a presence of a magnetic field from an MRI system.

In one embodiment, an implantable medical device configured to provide neurostimulation to a patient is disclosed herein. The device comprising a controller configured to control a stimulation parameter of the device, an MRI detecting system comprising a magnetic field sensing component, wherein the controller is configured to receive a signal from the MRI detecting system. The controller is configured to change the stimulation parameter of the device when the controller detects the signal from the MRI detecting system, wherein the signal is a voltage signal below a threshold indicating a presence of the magnetic field.

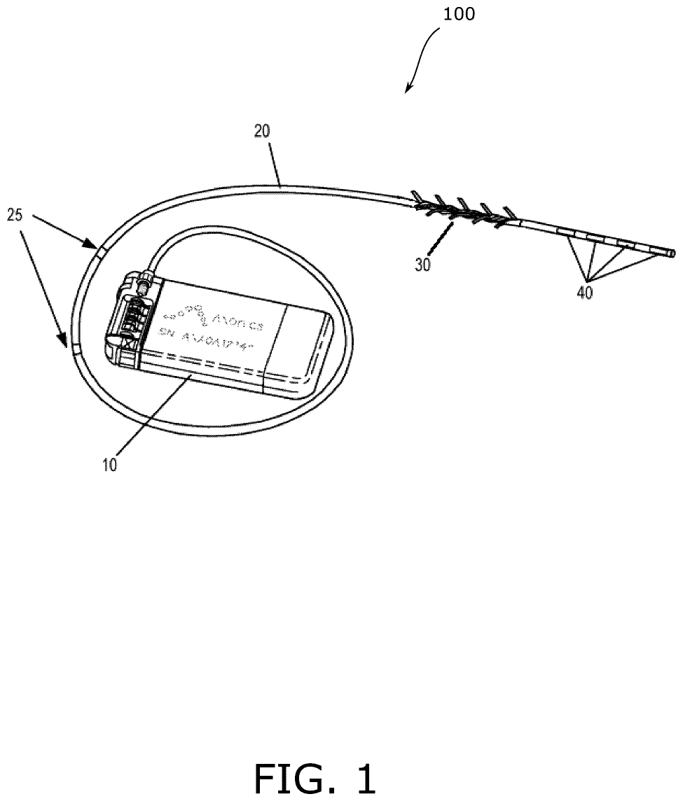

FIG. 1 illustrates an example neurostimulation system 100 that is fully implantable and adapted for sacral nerve stimulation treatment. The implantable system 100 includes an AIMD 10 that is coupled to a lead 20 that includes a group of electrodes 40 at a distal end of the lead. The lead includes a lead anchor portion 30 with a series of tines extending radially outward so as to anchor the lead and maintain a position of the lead 20 after implantation. The lead 20 may further include one or more radiopaque markers 25 to assist in locating and positioning the lead using visualization techniques such as fluoroscopy.

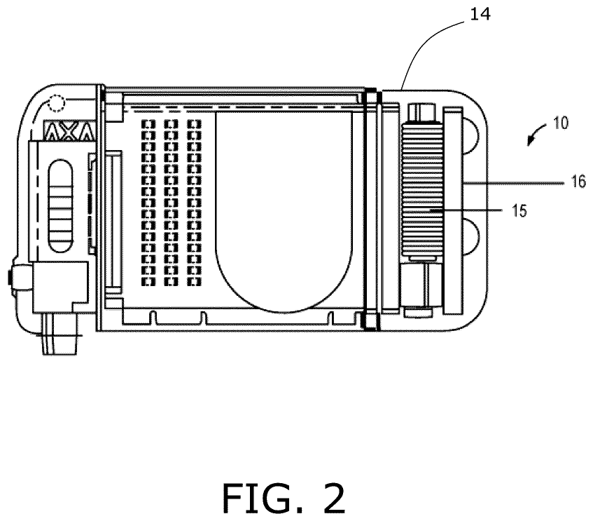

As shown in FIG. 2, the AIMD may include a case 14 which houses an antenna assembly 16 to facilitate wireless communication with a clinician programmer, a patient remote, and/or a charging coil to facilitate wireless charging with a wireless charger. The case may also house a charging coil assembly 15. The charging coil assembly includes a ferrite core.

This charging coil 15 may be utilized to identify an MRI environment and automatically switch the device to an MRI safe mode. The system described herein exploits the magnetic saturation of a ferromagnetic material (e.g., the charging coil ferrite core) when the device is in a strong magnetic field. As previously described, when the material is magnetically saturated, the permeability is largely reduced, resulting in a decreased inductance for the coil surrounding the ferromagnetic material. A suitable circuit may be used to monitor the change in inductance and produce a signal to trigger the MRI mode.

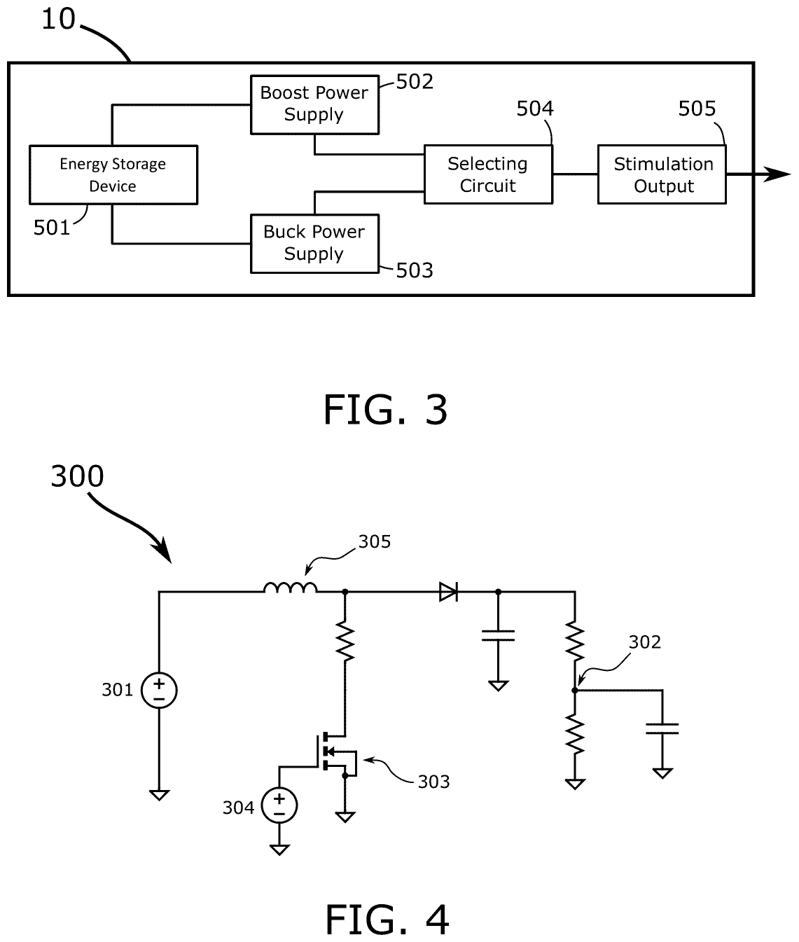

FIG. 3 shows a simplified schematic of an AIMD stimulation circuitry 500. The AIMD may include an energy source such as an energy storage device 501. The energy storage device 501 provides power to a boost power supply 502 and/or a buck power supply 503. The selecting circuit 504 is configured to select one of the boost power supply 502 and buck power supply 503 for the generation of the stimulation pulse for the stimulation output 505. The stimulation output 505 may be connected to the lead 20 as shown in FIG. 1. The boost power supply 502 may include a boost converter circuit (not shown). The boost converter circuit may include at least one inductor susceptible to saturation from the magnetic waves emitted from the MRI machine. The function of the boost converter is to increase the output voltage from the energy storage device 501 for use in therapy stimulation. The performance of the boost circuit can indicate whether or not a magnetic field is present near the AIMD. When a magnetic field is present, the performance of the boost converter circuit will be reduced (e.g., the voltage boost may have a lower magnitude for a given output load). Monitoring this performance will allow the AIMD to detect the presence of an MRI and allow the AIMD to switch to an MRI safe mode when the converter circuit performance decreases below a threshold.

The AIMD may include a magnetic field sensing circuit 300. For example, FIG. 4 shows an exemplary circuit 300 utilizing an inductor 305, which may be a dedicated inductor configured solely for sensing magnetic fields. However, in other embodiments the inductor 305 may be the charging coil 15 or the inductor found in the boost power supply 502. The dedicated inductor (e.g., a secondary minor winding not part of the charging coil or boost circuit) may be specifically configured to provide inductance and/or core-permeability information to be monitored by the circuit. The dedicated inductor may utilize the same core as the charging coil or boost circuit coil. This dedicated inductor may be smaller in size than the charging coil and/or the inductor found in the boost power supply. The sensing circuit 300 may be used to measure the inductance across the coil 305. The circuit includes a switch 303, which may be a transistor type element. When the switch 303 is closed, the inductor 305 stores energy due to the current provided by the power supply 301 that is carried through the inductor 305. The magnetic energy (E) stored in the inductor 305 may be determined from the following equation: E=½LI², where I is the charging current from the power supply 301, and L is the inductance of inductor 305. When the switch 303 is open, the stored energy in inductor 305 would be discharged, resulting in a measurable voltage at the node 302. When the inductance L of inductor 305 decreases due to the external magnetic field (e.g., produced by an MRI scan), the stored magnetic field in 305 also decreases, resulting in a smaller voltage at the sensing node 302. Measurement and monitoring of this sensing node 302 voltage may be used to trigger MRI mode. The voltage source 304 serves as a control for the switch 303 to control a duty cycle for the circuit 300.

As mentioned above, different components within the AIMD may contain ferromagnetic material. As an alternative or in addition to the charging coil or the inductor of boost converter circuit of the boost power supply, other inductors with ferromagnetic material in the AIMID may be exploited for inductance measurement and magnetic field (e.g., MRI) detection such as a dedicated inductor (e.g., a secondary minor winding not part of the charging coil) specifically configured to provide inductance and/or core-permeability information to be monitored by the circuit. The dedicated inductor may utilize the same core as the charging coil or boost circuit coil. This dedicated inductor may be smaller in size than the charging coil and/or the inductor found in the boost power supply. Each of these inductors may be incorporated into the sensing circuit shown in FIG. 4. Thus, the MRI detection system may utilize one or more of the sensing mechanisms and sensors that are described herein. If at least one of the sensors (or sensing circuits) indicates the presence of a magnetic field (e.g., due to the presence of an MRI scan) a controller in the AIMD may take action to adjust the operation of the AIMD.

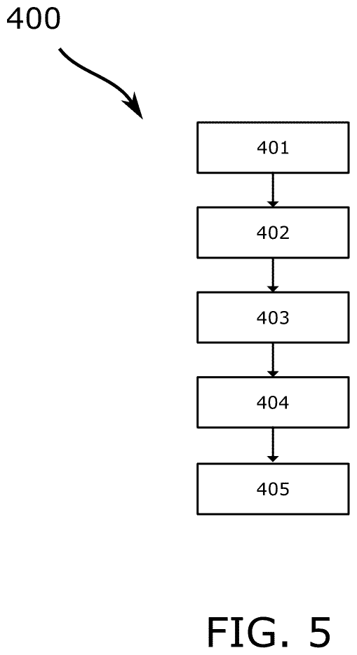

FIG. 5 shows exemplary steps 400 for an MRI detecting system. The inductance sensing circuit as shown in FIG. 3 may utilize the described steps thereof. The first step 401 is a measuring step. The inductance sensing circuit may indirectly measure the inductance value of the inductor 305 at predetermined time intervals (e.g. 5 seconds) at step 401 via the signal at point 302. Additional variables may also be used when any of the magnetic field sensing systems described herein is utilized. Accelerometer data regarding the patient's pose may be considered while measuring the inductance. For example, the accelerometer provides additional data to the AIMD in order to provide an MRI safe mode only when the patient is lying down. This would prevent false positives of MRI detection if, for example, a patient is subject to signals from a theft detector or a metal detector while standing.

At the second step 402, the measured signal is compared to a threshold. The third step 403 is a triggering step. For example, at step 402, when the measured signal representative of the inductance value of the inductor is lower than a threshold within a number of measurements, the system may trigger an MRI safe mode for the AIMD at step 403. The MRI safe mode may include adjustments to one or more stimulation parameters that differ from the normal parameters at the normal therapeutic setting, which can include turning off the stimulation completely, while the MRI is performed. The fourth step 404 is a monitoring step. For example, at step 404, the inductance sensing circuit 300 continues to monitor the inductance of the inductor 305. Once the sensed inductance surpasses the threshold, the sensing circuit will persistently monitor the signal until it remains continuously above the threshold for a predetermined duration. If the signal remains above the threshold for the predetermined duration, at step 405 the therapeutic stimulation may be reactivated while switching off the MRI mode. The threshold may be predetermined by the AIMD. In an alternative embodiment, the threshold may be calculated and, as a result, may change during operation of the AIMD, depending on variables such as stimulation parameters, AIMD structure, and/or patient device stimulation interface. Stimulation parameters may include stimulation frequency, stimulation amplitude, pulse width, duty cycle, and electrode configuration. AIMD structure may include size, shape, battery type, circuitry, material composition. The patient device stimulation interface may include, stimulation type (e.g. DBS/SCS/PNS), IPG placement or location, IPG orientation, lead placement or location, and stimulation location.

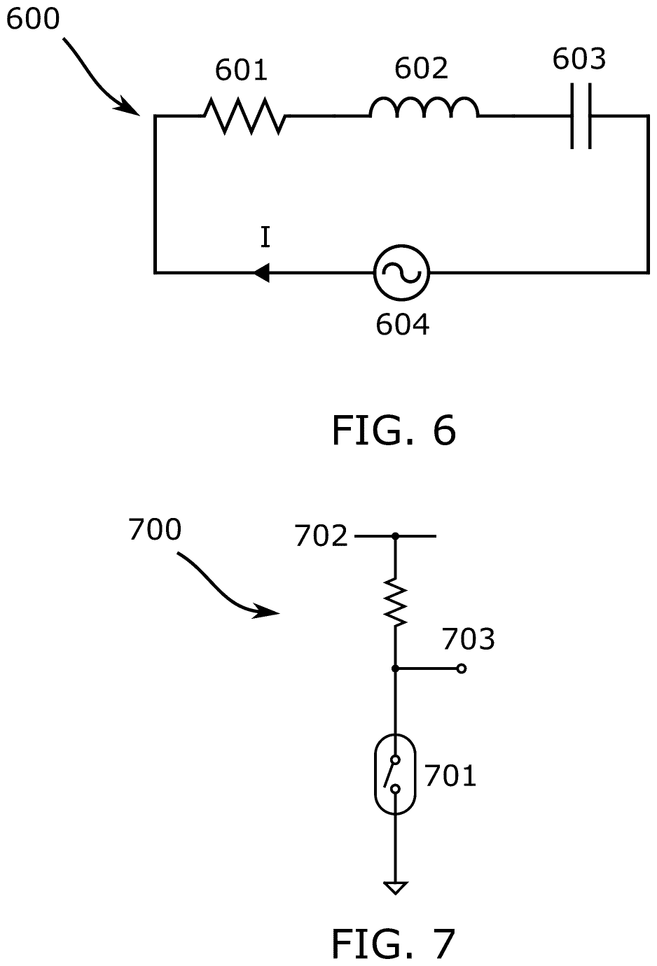

Other circuits may be provided in the AIMD to detect the magnetic field or the change of the magnetic field of the MRI. For example, as shown in FIG. 6 a resistor-inductor-capacitor (RLC) circuit 600 that is capable of detecting external magnetic fields (e.g., from an MRI) may be provided. The circuit 600 may include a resistor 601, inductor 602 and a capacitor 603. The inductor 602 may be any inductor described above such as a charging coil, a coil in the boost circuit or a dedicated inductor (e.g., a secondary minor winding separate from the charging coil and boost circuit) specifically configured to provide inductance and/or core-permeability information to be monitored by the circuit. The buck supply may also be utilized similarly, where the inductor of the buck supply is monitored. The dedicated inductor may utilize the same core as the charging coil or boost/buck circuit coil. This dedicated inductor may be smaller in size than the charging coil and/or the inductor found in the boost power supply. The inductance of inductor 602 may be predetermined and accompanied with a selected capacitor and resistor with predetermined capacitance and resistance, respectively. At a given frequency from AC input 604, the resonance frequency of the circuit may be calculated with the equation fᵣ=1/2π\sqrt{LC}. The behavior of an RLC circuit depends on the values of its components (e.g., resistor, inductor, and capacitor) and the frequency of the input signal from an AC input 604. When the signal from the input 604 is emitted at the frequency fᵣ (e.g. during therapy stimulation), then the circuit is at its resonance configuration. During circuit resonance the inductive reactance and capacitive reactance are equal and opposite, resulting in a net impedance of zero across the inductor and the capacitor, thus at resonance, impedance of the circuit is at its minimum. Deviation from the resonance frequency fᵣ would result in a lower current reading in the circuit, relative to its resonance configuration. When the circuit is subject to a strong magnetic field (e.g., from an MRI), the inductance of inductor 602 changes, which results in shift in the circuit's resonant frequency fᵣ. Since the frequency of AC input 604 was at a predetermined frequency when inductance of the inductor 602 was at its highest value (e.g., non-saturated state), the presence of the MRI would result in a lower current reading, relative to the current during the circuit's resonant configuration. This state may be monitored by evaluating the voltage drop across the resistor 601. Since the net impedance of the inductor 602 and capacitor 603 will increase during the non-resonant state during MRI interference, voltage across resistor 601 will decrease (see Kirchhoff's Voltage Law). When voltage across the resistor 601 falls below a voltage threshold, the MRI mode of the AIMD may be activated.

The voltage threshold may be predetermined by the AIMD. In an alternative embodiment, the threshold may be calculated and, as a result, may change during operation of the AIMD, depending on variables such as stimulation parameters, AIMD structure, and/or patient device stimulation interface. Stimulation parameters may include stimulation frequency, stimulation amplitude, pulse width, duty cycle, and electrode configuration. AIMD structure may include size, shape, battery type, circuitry, material composition. Patient device stimulation interface may include, stimulation type (e.g. DBS/SCS/PNS), IPG placement or location, IPG orientation, lead placement or location, and stimulation location.

In another embodiment, a reed switch may be utilized to detect presence of an MRI. An exemplary circuit 700 including a reed switch 701 and input voltage 702 is shown in FIG. 7. When a magnetic field is present, the reed switch 701 will close, which causes a voltage signal to be present at node 703. The voltage signal may be a trigger for the AIMD to activate an MRI safe mode.

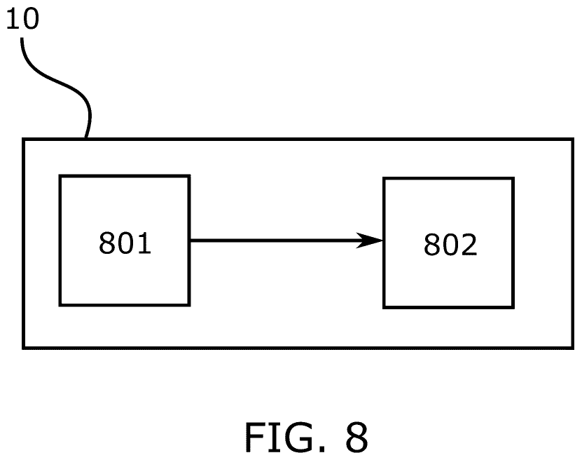

In another embodiment, a Hall effect sensor may be utilized and continuously monitored to detect the presence of the MRI. As shown in FIG. 8, a microcontroller 802 may monitor the output signal from Hall sensor 801. Since the output signal from Hall sensor 801 varies as a function of a sensed magnetic field, the microcontroller 802 may communicate with the AIMD when the output signal falls below a threshold in order to activate an MRI safe mode for the AIMD 10.

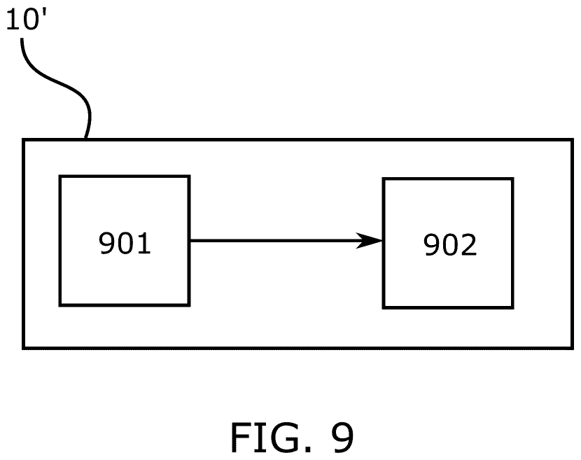

In another embodiment, a magneto resistive sensor may be utilized in order to detect the presence of the MRI. As shown in FIG. 9, a microcontroller 902 may monitor one or more output signals from a magneto resistive sensor 901 comprising one or more MR sensing circuit(s) for the AIMD 10. Magneto resistive (MR) sensor 901 may include one or more magnetic field detection devices that experience changes in electrical resistance in response to an external magnetic field. MR sensor 901 may be an anisotropic magnetoresistance (AMR), giant magnetoresistance (GMR), and/or tunneling magnetoresistance (TMR) sensor. AMR, GMR, and TMR sensors are magneto resistive and thus can be monitored to detected changes in electrical resistance when the sensors are subjected to a magnetic field. These MR sensors generally consume low power, which makes them suitable for long term AIMD operation. Any of the above described MR sensor may be incorporated into a portion of the circuitry of the AIMD 10.

In certain embodiments described above, it may not be possible to activate an MRI safe mode. The feasibility of an MRI safe mode can vary depending on the characteristics of the AIMD. Some devices may not be able to achieve an MRI safe mode. In an MRI safe mode, the AIMD can undergo an MRI scan without considering specific MRI characteristics. As an alternative for some AIMDs, an MRI conditional mode may be activated. When operating in MRI conditional mode, the AIMD's eligibility for an MRI scan is contingent on factors such as magnetic field strength, spatial gradient, time-varying magnetic fields, radio frequency fields, specific absorption rate, and AIMD configurations (e.g., lead type or routing).

Although described as alternative embodiments, any of the solutions described above may be utilized together in combination to provide a redundant system. Having two (or more) magnetic sensing solutions may provide more accurate detection of an MRI.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations disclosed herein are not intended to be limited to the particulars disclosed herein; rather, implementations of the present invention extend to all functionally equivalent structures, methods and uses, such as are within the scope of the appended claims.

What is claimed is:

1. An implantable medical device configured to provide electrical stimulation to a patient, the device comprising:
a rechargeable energy storage device;
a charging coil configured to receive energy to recharge the rechargeable energy storage device;
a controller configured to control a stimulation parameter of the device;
an MRI detecting circuit electrically connected to the charging coil, wherein the controller is configured to receive a signal from MRI detecting circuit;
wherein the controller is configured to change the stimulation parameter of the device when the controller detects the signal from the MRI detecting circuit, wherein the signal representative of an inductance change of the charging coil due to a presence of a magnetic field from an MRI system.

2. The device of claim 1, wherein the signal is an electrical signal representative of an inductance of the charging coil below an inductance threshold.

3. An implantable medical device configured to provide neurostimulation to a patient, the device comprising:
a controller configured to control a stimulation parameter of the device;
an MRI detecting system comprising a magnetic field sensing component, wherein the controller is configured to receive a signal from the MRI detecting system;
wherein the controller is configured to change the stimulation parameter of the device when the controller detects the signal from the MRI detecting system, wherein the signal is a voltage signal below a threshold indicating a presence of the magnetic field.

4. The device of claim 3, further comprising:
a boost supply connected to the energy storage device;
wherein the MRI detecting system monitors the performance of the boost supply as part of the magnetic field sensing component.

5. The device of claim 4, further comprising:
wherein the boost supply is connected to a stimulation output of the device.

6. The device of claim 3, further comprising:
a buck supply connected to the energy storage device;
wherein the MRI detecting system monitors the performance of the buck supply as part of the magnetic field sensing component.

7. The device of claim 6, further comprising:
wherein the buck supply is connected to a stimulation output of the device.

8. The device of claim 3, further comprising a second magnetic field sensing component, wherein the MRI detecting system is configured to send the signal to the controller when at least one of the magnetic field sensing components senses the presence of a magnetic field.

9. An implantable medical device configured to provide electrical stimulation to a patient, the device comprising:
a rechargeable energy storage device;
a charging coil configured to receive energy to recharge the rechargeable energy storage device;
a controller configured to control a stimulation parameter of the device;
a first magnetic field detecting component, wherein the controller is configured to receive a first signal from the first magnetic field detecting component;
a second magnetic field detecting component, wherein the controller is configured to receive a second signal from the second magnetic field detecting component; and
wherein the controller is configured to change the stimulation parameter of the device when the controller detects at least one of the first signal and the second signal, wherein the first signal is representative of an inductance change of the charging coil due to a presence of a magnetic field from an MRI system and wherein the second signal is a voltage signal below a threshold indicating the presence of the magnetic field.

10. The device of claim 9, wherein the controller is configured to periodically monitor the signals from the first and second magnetic field detecting components.

11. The device of claim 9, further comprising:
a boost supply connected to the energy storage device;
wherein the boost supply communicates with the first magnetic field detecting component, such that the performance of the boost supply is monitored as part of the first signal.

12. The device of claim 11, further comprising:
wherein the charging coil communicates with the second magnetic field detecting component.

13. The device of claim 9, further comprising:
a buck supply connected to the energy storage device;
wherein the buck supply is part of the first magnetic field detecting component, such that the performance of the buck supply is monitored as part of the first signal.

14. The device of claim 13, further comprising:
wherein the charging coil communicates with the second magnetic field detecting component.

15. An implantable medical device configured to provide electrical stimulation to a patient, the device comprising:
a rechargeable energy storage device;
a charging coil configured to receive energy to recharge the rechargeable energy storage device;
a controller configured to control a stimulation parameter of the device;
a boost circuit connected to the energy storage device, wherein the boost circuit is configured to increase output voltage from the energy storage device;
an MRI detecting circuit electrically connected to the boost circuit, wherein the controller is configured to receive a signal from MRI detecting circuit;
wherein the controller is configured to change the stimulation parameter of the device when the controller detects the signal from the MRI detecting circuit, wherein the signal is representative of an electrical property change of the boost circuit due to a presence of a magnetic field from an MRI system.

16. The device of claim 15, further comprising, wherein the MRI detecting circuit also monitors the performance of the charging coil.

17. The device of claim 15, further comprising, wherein the electrical property change is representative of inductance crossing a threshold inductance.

18. The device of claim 15, further comprising, wherein the electrical property change is representative of voltage crossing a threshold voltage.

19. The device of claim 15, further comprising:
a buck supply connected to the energy storage device, wherein the boost circuit is configured to decrease output voltage from the energy storage device;
wherein the MRI detecting system monitors the performance of the buck supply as part of the magnetic field sensing component.
